(19) United States
(12) Patent Application Publication
(10) Pub. No.: US 2019/0254510 A1
(43) Pub. Date: Aug. 22, 2019

(54) ENDOSCOPE LIGHT SOURCE DEVICE, ENDOSCOPE, AND ENDOSCOPE SYSTEM

(71) Applicant: HOYA CORPORATION, Tokyo (JP)

(72) Inventor: Kunihiko ONOBORI, Tokyo (JP)

(73) Assignee: HOYA CORPORATION, Tokyo (JP)

(21) Appl. No.: 16/308,453

(22) PCT Filed: Aug. 30, 2017
(86) PCT No.: PCT/JP2017/031145

§ 371 Date: Dec. 7, 2018

(30) Foreign Application Priority Data
Aug. 31, 2016 (JP) 2016-169504

(51) Int. Cl. A61B 1/06; A61B 1/00; A61B 1/04; A61B 1/07; G02B 23/24

(52) U.S. Cl. CPC A61B 1/0638; A61B 1/00096; A61B 1/04; G02B 23/26; A61B 1/0676; G02B 23/2461; A61B 1/00009; A61B 1/07

(57) ABSTRACT

A light source unit of a light source device for an endoscope, which makes it possible to improve light use efficiency regarding illumination light without upsizing an optical system, includes: a solid-state light emitting element for emitting light from a light emitting surface; and a cover member that covers the solid-state light emitting element such that the cover member is spaced apart from the light emitting surface. In this configuration, the cover member includes a reflective surface that reflects light emitted from the light emitting surface, and an opening from which light is emitted.

### TECHNICAL FIELD

The present invention relates to an endoscope light source device for irradiating an object with light, an endoscope, and an endoscope system.

### BACKGROUND ART

There are known light sources for endoscopes that use solid-state light emitting elements, such as LEDs (Light Emitting Diodes) and LDs (Laser Diodes), instead of xenon lamps. For example, Patent Document 1 discloses an endoscope device that includes a white LED and a purple LED. In the endoscope device of Patent Document 1, light emitted from each LED is condensed by a lens, and enters an optical fiber of the electronic endoscope. The light that entered the optical fiber exits from a distal end portion of the electronic endoscope and illuminates the subject.

### PRIOR ART DOCUMENTS

### Patent Documents

Patent Document 1: WO 2012/108420

### SUMMARY OF INVENTION

### Problem to be Solved by Invention

The endoscope device described in Patent Document 1 uses LEDs as the light source device. LED light emitted from LEDs has a radial light intensity distribution (lambert distribution). Therefore, in order to improve the light use efficiency of the endoscope device regarding LED light, the optical system needs to be upsized so as to be able to capture light with a large emission angle. However, as a result of the optical system being upsized, the light source device is upsized, and therefore it is difficult to realize a compact endoscope device.

The present invention has been achieved in light of the above-described circumstances, and an object of the present invention is to provide an endoscope light source device, an endoscope, and an endoscope system that are capable of improving light use efficiency regarding illumination light without upsizing an optical system.

### Means to Solve Problem

To solve the above-described problem, an endoscope light source device according to one embodiment of the present invention includes: a solid-state light emitting element configured to emit light from a light emitting surface thereof and a cover member that covers the solid-state light emitting element such that the cover member is spaced apart from the light emitting surface. The cover member includes an opening configured to emit some of the light emitted from the light emitting surface and some of the reflected light reflected by the reflective surface.

According to this configuration, in the light emitted from the solid-state light emitting element, light emitted toward the opening can be taken out of the opening. Also, in the light emitted from the solid-state light emitting element, light emitted in directions other than a direction toward the opening is reflected by the reflective surface of the cover member and converted to light that is directed toward the opening. In this way, the distribution of emission angles of light emitted from the light source device becomes narrower than the distribution of emission angles of light emitted from the solid-state light emitting element, and therefore light use efficiency can be improved without upsizing an optical system.

According to one embodiment of the present invention, the area of the opening is preferably smaller than the area of the light emitting surface.

According to one embodiment of the present invention, the cover member preferably has a hollow dome shape.

According to one embodiment of the present invention, preferably, the cover member includes a light-transmitting substrate configured to allow the light emitted from the light emitting surface to pass therethrough, the reflective surface is a region of a surface of the light-transmitting substrate where a reflective film configured to reflect the light emitted from the light emitting surface is formed, and the opening is a region of the surface of the light-transmitting substrate where the reflective film is not formed.

According to one embodiment of the present invention, the endoscope light source device preferably includes a convex lens disposed in the opening.

According to one embodiment of the present invention, the cover member preferably includes a plano-convex lens disposed such that a flat surface thereof is opposite the light emitting surface. In this configuration, preferably, the reflective surface is a region of a convex surface of the plano-convex lens where a reflective film is formed, and the opening is a region of the convex surface where the reflective film is not formed.

According to one embodiment of the present invention, the endoscope light source device includes a protective member that protects the light emitting surface. In this configuration, preferably, the protective member is disposed to cover the light emitting surface and configured to allow the light emitted from the light emitting surface to pass therethrough, and the cover member is disposed to cover the solid-state light emitting element and the protective member.

According to one embodiment of the present invention, the endoscope light source device preferably includes a phosphor disposed between the light emitting surface and the reflective surface and configured to absorb some of the light emitted from the light emitting surface and emit fluorescent light.

According to one embodiment of the present invention, the phosphor is preferably disposed on the light emitting surface.

An endoscope system according to one embodiment of the present invention includes:

an endoscope light source device that includes a first light source unit including a first solid-state light emitting element configured to emit first light from a first light emitting surface, and a first cover member that covers the first solid-state light emitting element such that the first cover member is spaced apart from the first light emitting surface, the first cover member including a first reflective surface configured to reflect the first light emitted from the first light emitting surface and a first opening configured to emit some of the first light and some of the reflected light reflected by the first reflective surface; and

an endoscope that includes a connection portion connected to the endoscope light source device and a distal end portion having an illumination light emission opening configured to emit illumination light transmitted through an optical cable from the connection portion to illuminate a subject.

In the endoscope system according to one embodiment of the present invention, it is preferable that

the endoscope light source device includes, in addition to the first light source unit,

a second light source unit including a second solid-state light emitting element configured to emit second light from a second light emitting surface thereof, a second cover member that covers the second solid-state light emitting element such that the second cover member is spaced apart from the second light emitting surface, the second cover member including a reflective surface configured to reflect the second light and an opening configured to emit some of the second light and some of the reflected light reflected by the second reflective surface, and the second light source unit including a phosphor disposed between the second light emitting surface and the second reflective surface and configured to absorb some of the second light and emit fluorescent light; and

an optical element provided on a light path of the first light and a light path of the second light and the fluorescent light and configured to extract the fluorescent light from the second light and the fluorescent light and emit combined light on a light path that combines the light path of the first light and the light path of the fluorescent light.

In the endoscope system according to one embodiment of the present invention, it is preferable that the phosphor includes material configured to emit a light component having a wavelength band of 460 to 600 nm.

An endoscope according to one embodiment of the present invention includes: a light source unit including a solid-state light emitting element configured to emit light from a light emitting surface thereof, and a cover member that covers the solid-state light emitting element such that the cover member is spaced apart from the light emitting surface, the cover member including a reflective surface configured to reflect the light emitted from the light emitting surface, and an opening configured to emit some of the light and some of the reflected light reflected by the reflective surface, and a distal end portion having an illumination light emission opening configured to emit the light emitted by the light source unit as illumination light to illuminate a subject.

### Effect of Invention

With the endoscope light source device and the endoscope system described above, it is possible to improve light use efficiency regarding illumination light without upsizing an optical system.

### BRIEF DESCRIPTION OF DRAWINGS

FIG. 1 is a block diagram showing a configuration of an electronic endoscope system that includes an endoscope light source device according to a first embodiment of the present invention.

FIG. 2 is a block diagram of the light source device according to one embodiment of the present invention.

FIG. 3 is a diagram showing a spectral intensity distribution of illumination light emitted from light source units according to one embodiment of the present invention.

FIG. 4 is a perspective view of a first light source unit according to one embodiment of the present invention.

FIG. 5 is a cross sectional view of the first light source unit according to one embodiment of the present invention.

FIG. 6 is a cross sectional view of a third light source unit according to one embodiment of the present invention.

FIG. 7 is a cross sectional view of a first light source unit according to one embodiment of the present invention.

FIG. 8 is a diagram showing spectral intensity distribution of light emitted from a third light source unit according to one embodiment of the present invention.

FIG. 9 is a cross sectional view of a first light source unit according to one embodiment of the present invention.

FIG. 10 is a cross sectional view of a first light source unit according to one embodiment of the present invention.

FIG. 11 is a cross sectional view of a first light source unit according to one embodiment of the present invention.

FIG. 12 is a cross sectional view of a first light source unit according to one embodiment of the present invention.

### DESCRIPTION OF EMBODIMENTS

Hereinafter, one embodiment of the present invention will be described with reference to the drawings. Note that an electronic endoscope system that includes an endoscope light source unit is taken as an example of an embodiment of the present invention in the following description.

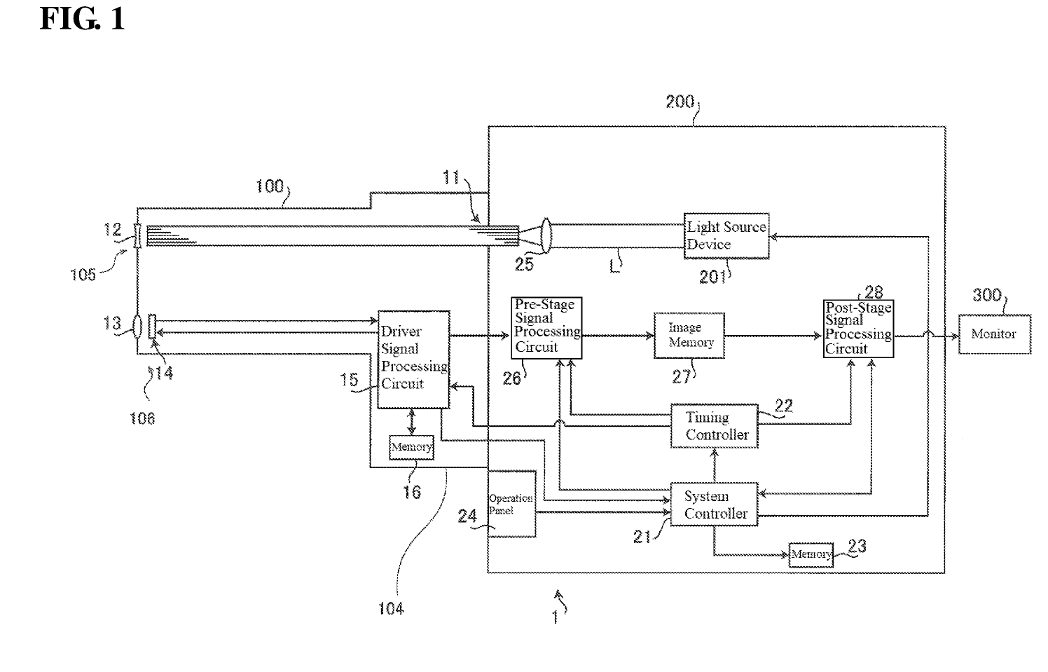

FIG. 1 is a block diagram showing the configuration of an electronic endoscope system 1 that includes an endoscope light source device 201 according to an embodiment of the present invention. As shown in FIG. 1, the electronic endoscope system 1 is a system specialized for medical use, and includes an electronic endoscope (endoscope) 100, a processor 200, and a monitor 300.

The processor 200 includes a system controller 21 and a timing controller 22. The system controller 21 executes various programs stored in a memory 23 and performs overall control of the electronic endoscope system 1. Also, the system controller 21 is connected to an operation panel 24. The system controller 21 changes various operations of the electronic endoscope system 1 and parameters for various operations in accordance with instructions from an operator that are input using the operation panel 24. The timing controller 22 outputs a clock pulse, which is for adjustment of the timing of the operations of portions, to circuits in the electronic endoscope system 1.

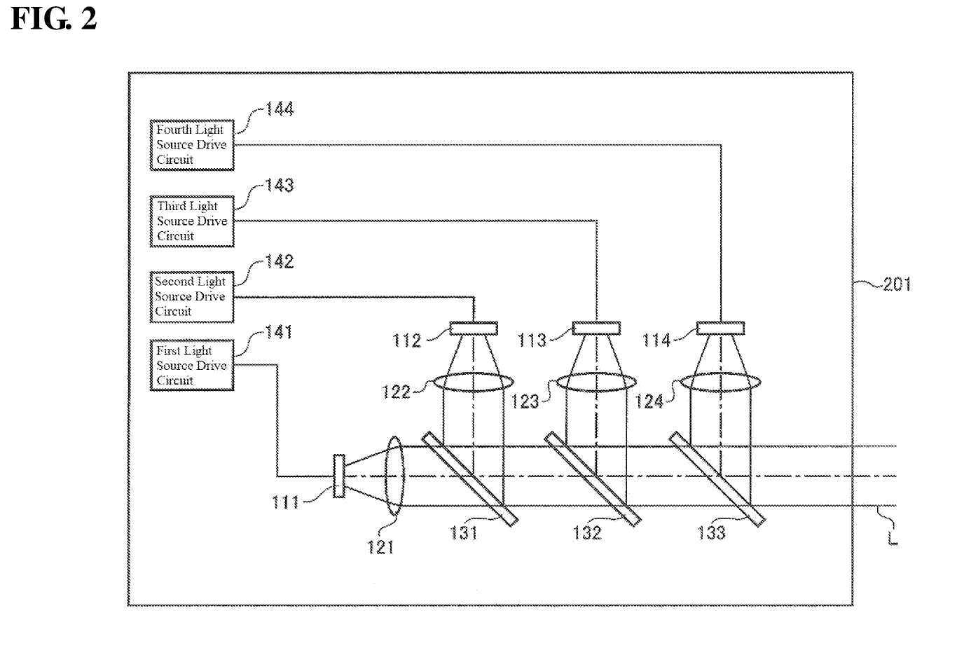

The processor 200 includes a light source device 201. FIG. 2 is a block diagram showing the configuration of the light source device 201. The light source device 201 includes first to fourth light source units 111 to 114. The emission of light by the first to fourth light source units 111 to 114 is controlled by first to fourth light source drive circuits 141 to 144, respectively.

The first light source unit 111 is a purple light emitting diode (LED: Light Emitting Diode) that emits light in the purple wavelength band (e.g., wavelengths of 395 to 435 nm). The second light source unit 112 is a blue LED that emits light in the blue wavelength band (e.g., wavelengths of 430 to 470 nm). The third light source unit 113 has a blue LED that emits light in the blue wavelength band (e.g., wavelengths of 425 to 455 nm) and a phosphor. The phosphor is excited by blue LED light emitted from the blue LED and emits fluorescent light in the green wavelength band (e.g., wavelengths of 460 to 600 nm). The fourth light source unit 114 is a red light emitting diode that emits light in the red wavelength band (e.g., wavelengths of 620 to 680 nm).

Collimator lenses 121 to 124 are arranged in front of, with respect to the light emission direction, the light source units 111 to 114, respectively. The purple LED light emitted from the first light source unit 111 is converted into parallel light by the collimator lens 121 and is then incident on a dichroic mirror 131. Also, the LED light emitted from the second light source unit 112 is converted into parallel light by the collimator lens 122 and is then incident on the dichroic mirror 131. The dichroic mirror 131 combines the light path of the light emitted from the first light source unit 111 and the light path of the light emitted from the second light source unit 112. Specifically, the dichroic mirror 131 has a cutoff wavelength of approximately 430 nm, and has a characteristic of allowing the passage of light with a shorter wavelength than the cutoff wavelength and reflecting light with a wavelength greater than or equal to the cutoff wavelength. For this reason, the purple LED light emitted from the first light source unit 111 passes through the dichroic mirror 131, and the blue LED light emitted from the second light source unit 112 is reflected by the dichroic mirror 131. Accordingly, the light paths of the purple LED light and the blue fluorescent light are combined with each other. The light on the light paths combined by the dichroic mirror 131 is incident on a dichroic mirror 132.

Also, the light emitted from the third light source unit 113, that is to say blue LED light and green fluorescent light, is converted into parallel light by the collimator lens 123 and is then incident on the dichroic mirror 132. The dichroic mirror 132 combines the light path of light from the dichroic mirror 131 and the light path of light emitted from the third light source unit 113. Specifically, the dichroic mirror 132 has a cutoff wavelength of approximately 500 nm, and has a characteristic of allowing the passage of light with a shorter wavelength than the cutoff wavelength and reflecting light with a wavelength greater than or equal to the cutoff wavelength. For this reason, the light path of the purple LED light and the blue LED light from the dichroic mirror 131 and the light path of the green fluorescent light included in the light emitted from the third light source unit 113 are combined by the dichroic mirror 132. The light on the light paths combined by the dichroic mirror 132 is incident on a dichroic mirror 133.

Also, the red LED light emitted from the fourth light source unit 114 is converted into parallel light by the collimator lens 124 and is then incident on the dichroic mirror 133. The dichroic mirror 133 combines the light path of the light from the dichroic mirror 132 and the light path of the red LED light emitted from the fourth light source unit 114. Specifically, the dichroic mirror 133 has a cutoff wavelength of approximately 600 nm, and has a characteristic of allowing the passage of light with a shorter wavelength than the cutoff wavelength and reflecting light with a wavelength greater than or equal to the cutoff wavelength. For this reason, the light path of the light from the dichroic mirror 132 and the light path of the red LED light emitted from the fourth light source unit 114 are combined by the dichroic mirror 133, and the light on the combined light paths is emitted from the light source device 201 as illumination light L.

The illumination light L emitted from the light source device 201 is condensed on the entrance surface of an LCB (Light Carrying Bundle) 11 by a condensing lens 25, and enters the LCB 11.

The illumination light L that entered the LCB 11 propagates inside the LCB 11. The illumination light L that propagated inside the LCB 11 exits from the exit surface of the LCB 11 arranged at an distal end portion 106 of the electronic endoscope 100 and passes through a light distribution lens 12, and then illuminates the subject. Returning light from the subject, which was illuminated by the illumination light L from the light distribution lens 12, passes through an objective lens 13 and forms an optical image on the light receiving surface of a solid-state image sensor 14.

The solid-state image sensor 14 is a single-plate color CCD (Charge Coupled Device) image sensor that has a Bayer pixel arrangement. The solid-state image sensor 14 accumulates charge according to the light quantity of an optical image formed on pixels on the light receiving surface, generates R (Red), G (Green), and B (Blue) image signals, and outputs the image signals. Note that the solid-state image sensor 14 is not limited to being a CCD image sensor, and may be replaced with a CMOS (Complementary Metal Oxide Semiconductor) image sensor or another type of imaging device. The solid-state image sensor 14 may also be an element that includes a complementary color filter.

A driver signal processing circuit 15 is provided in the connection portion of the electronic endoscope 100. An image signal regarding the subject is input from the solid-state image sensor 14 to the driver signal processing circuit 15 at a predetermined frame cycle. The frame cycle is 1/30 sec, for example. The image signal input from the solid-state image sensor 14 is subjected to predetermined processing by the driver signal processing circuit 15 and output to a pre-stage signal processing circuit 26 of the processor 200.

The driver signal processing circuit 15 also accesses a memory 16 and reads out unique information regarding the electronic endoscope 100. The unique information regarding the electronic endoscope 100 recorded in the memory 16 includes, for example, the pixel count, sensitivity, operable frame rate, and model number, etc., of the solid-state image sensor 14. The unique information read out from the memory 16 is output by the driver signal processing circuit 15 to the system controller 21.

The system controller 21 generates control signals by performing various computation based on the unique information regarding the electronic endoscope 100. The system controller 21 uses the generated control signals to control the operations of and the timing of the various circuits in the processor 200 so as to perform processing suited to the electronic endoscope 100, which is connected to the processor 200.

The timing controller 22 supplies a clock pulse to the driver signal processing circuit 15 in accordance with timing control performed by the system controller 21. In accordance with the clock pulse supplied from the timing controller 22, the driver signal processing circuit 15 controls the driving of the solid-state image sensor 14 at a timing synchronized with the frame rate of the images processed by the processor 200.

The pre-stage signal processing circuit 26 performs predetermined signal processing such as demosaicing processing, matrix computation, and Y/C separation, etc., on the image signal received in one frame cycle from the driver signal processing circuit 15, and outputs the result to an image memory 27.

The image memory 27 buffers image signals received from the pre-stage signal processing circuit 26, and outputs the image signals to a post-stage signal processing circuit 28 in accordance with timing control performed by the timing controller 22.

The post-stage signal processing circuit 28 performs processing on the image signals received from the image memory 27 to generate screen data for monitor display, and converts the generated monitor display screen data into a predetermined video format signal. The converted video format signal is output to the monitor 300. Accordingly, subject images are displayed on the display screen of the monitor 300.

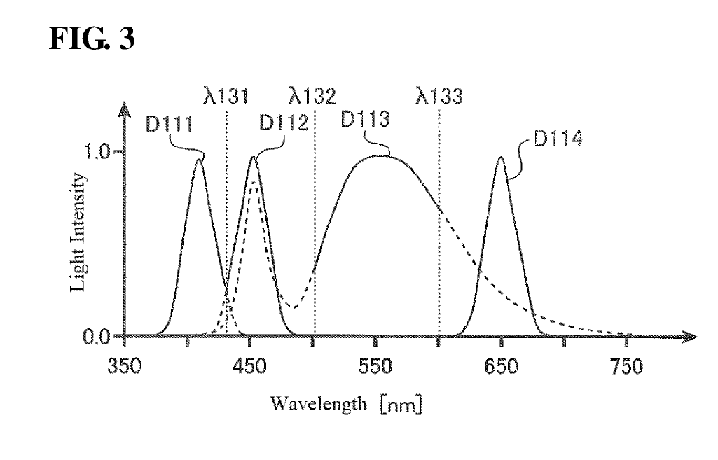

FIG. 3 shows the spectral intensity distributions D111 to 114 of the illumination light L emitted from the respective light source units 111 to 114. In FIG. 3, the horizontal axis in the spectral intensity distributions indicates the wavelength (nm), and the vertical axis indicates the intensity of the illumination light L. Note that the vertical axis is standardized such that the maximum intensity value is 1. In addition, in FIG. 3, the cutoff wavelengths fÉ131 to fÉ133 of the respective dichroic mirrors 131 to 133 are indicated by broken lines. In the spectral intensity distributions shown in FIG. 3, the regions indicated by solid lines are the regions emitted from the light source device 201 and used as the illumination light L. The regions indicated by broken lines are the regions not emitted from the light source device 201 and not used as the illumination light L.

As shown in FIG. 3, the light paths of light emitted from the light source units 111 to 114 are combined by the respective dichroic mirrors 131 to 133, and therefore the light source device 201 emits the illumination light L that has a wide wavelength range spanning from the ultraviolet region (part of the near ultraviolet region) to the red region. The spectral intensity distribution of this illumination light L is the combination of the regions indicated by solid lines in the spectral intensity distributions D111 to D114 shown in FIG. 3. Note that the light source units 111 to 114 can be independently controlled. For this reason, the intensities of the light emitted from the light source units 111 to 114 can be changed according to the subject.

Therefore, the endoscope system according to the first embodiment includes an electronic endoscope that includes a connection portion 104 (see FIG. 1) connected to the endoscope light source device 201, which is provided with the light source units 111, 112, and 114, and the distal end portion 106, which includes an illumination light emission opening 105 configured to emit the illumination light L transmitted through the LCB 11 (an optical cable) from the connection portion 104 to illuminate the subject.

According to the first embodiment, in addition to the light source units 111 and 112, the light source device 210 preferably includes a light source unit 113 including a phosphor 32 and a dichroic mirror 132 (an optical element) that is provided on the light paths of the light emitted from the first light source units 111 and 112 and on the light path of the light emitted from the first light source unit 113 and is configured to extract fluorescent light from the light emitted from the light source unit 113 and emit combined light on the light path that combines the light paths of the light emitted from the first light source units 111 and 112 and the light path of the fluorescent light. As the wavelength band of the fluorescent light emitted by the phosphor is wide, light having a wide wavelength band can be easily emitted. When generating pseudo white light using fluorescent light, as fluorescent light has lower light intensity than that of the light of other color components, illumination light of pseudo white light tends to be dim. For this reason, it is necessary to increase the low light intensity of fluorescent light. Therefore, conventionally, it is necessary to pass a large electric current through an LED to increase the emission intensity of the excitation light; according to the present embodiment, however, as the light intensity of the fluorescent light can be increased by providing a cover member 40 as described below, it is no longer necessary to pass a large electric current through the LED, which is preferable from the standpoint of energy consumption.

According to one embodiment, preferably, the phosphor includes material that emits a light component having a wavelength band of 460 to 600 nm. The light component in this wavelength band is the green component, which is a component that can be easily absorbed in a biological tissue in various narrower wavelength bands in this wavelength band. Therefore, it is desirable to increase the light intensity of a wavelength band of 460 to 600 nm to make it easier to discern the effect of absorption and non-absorption of light by a biological tissue.

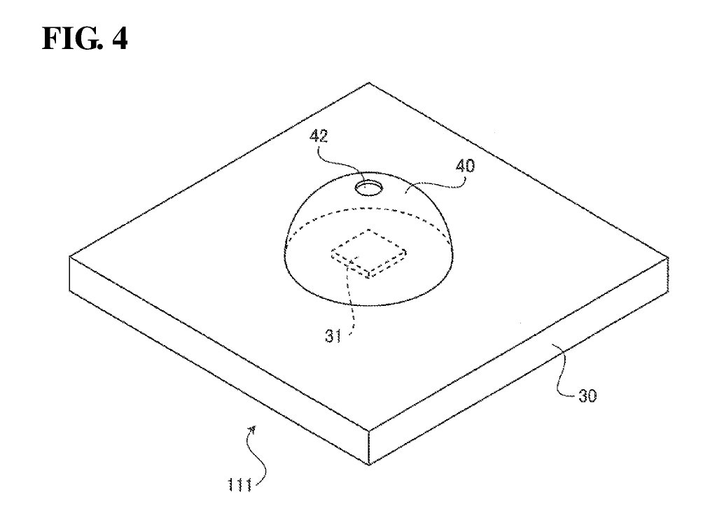

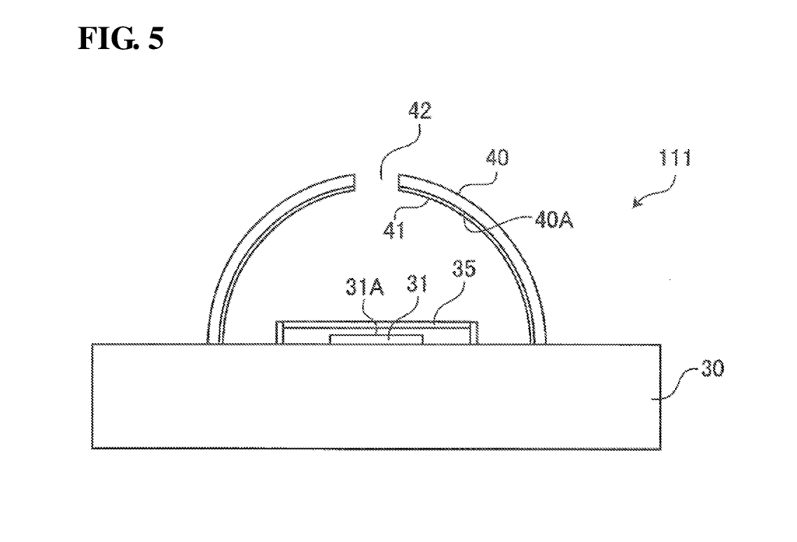

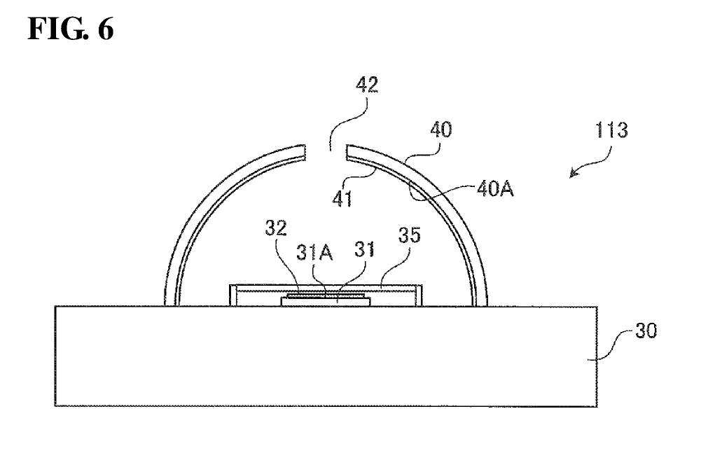

FIGS. 4 to 6 illustrate the configurations of the light source units 111 to 114. FIG. 4 is a perspective view of the first light source unit 111. FIG. 5 is a cross sectional view of the first light source unit 111. FIG. 6 is a cross sectional view of the third light source unit 113. The third light source unit 113 has the same configuration as the first light source unit 111 except that it includes a phosphor and that its LED has a different emission wavelength. Also, the second and fourth light source units 112 and 114 have the same configuration as the first light source unit 111 except that their LEDs have different emission wavelengths.

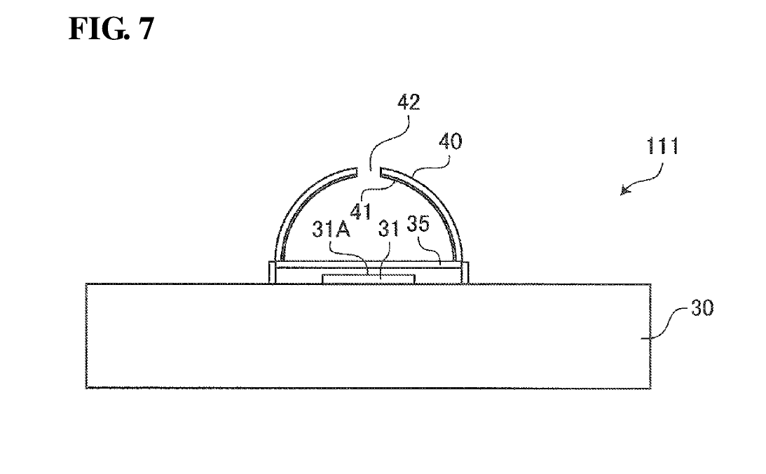

The first light source unit 111 includes a substrate 30, a solid-state light emitting element 31 mounted on the substrate 30, and a cover member 40. The solid-state light emitting element 31 includes a light emitting surface 31A that emits LED light. The solid-state light emitting element 31 emits light according to the drive current supplied via wiring (not shown) formed in the substrate 30. The first light source unit 111 includes a light-transmitting cover glass 35 for protecting the solid-state light emitting element 31. The cover member 40 includes a base plate that has a hollow dome shape (a hemispherical shell shape), and is disposed to cover the solid-state light emitting element 31 and be spaced apart from the light emitting surface 31A of the solid-state light emitting element 31. In the configurations shown in FIGS. 4 to 6, while the cover member 40 is disposed to cover the cover glass 35 and the solid-state light emitting element 31, the present embodiment is not limited to this configuration. FIG. 7 is a cross sectional view of the first light source unit 111 in a variation of the present embodiment. As in the first light source unit 111 shown in the FIG. 7, the cover member 40 may be disposed on the cover glass 35.

A reflective film 41 that reflects LED light or fluorescent light is formed on the inner wall surface 40A of the cover member 40. The reflective film 41 is, for example, a metallic (for example, silver) multilayer film or a dielectric multilayer film. The surface (the reflective surface) of the reflective film 41 has a relatively high reflectivity to LED light or fluorescent light. Also, an opening 42 is formed in a part of the region of the cover member 40 that is opposite the light emitting surface 31A of the solid-state light emitting element 31. The opening 42 is a through hole that connects the inside and the outside of the cover member 40. The area of the opening 42 is set to be smaller than the area of the light emitting surface 31A of the solid-state light emitting element 31.

The cover member 40 of the first light source unit 111 is used to improve the light use efficiency, regarding LED light, of the optical system that uses the collimator lens 121. In the LED light emitted from the solid-state light emitting element 31, LED light emitted toward the opening 42 of the cover member 40 passes through the opening 42, and is thus emitted from first light source unit 111. Meanwhile, in the LED light emitted from the solid-state light emitting element 31, the LED light emitted in directions other than a direction toward the opening 42 is reflected by the reflective surface of the cover member 40 toward the substrate 30 or the solid-state light emitting element 31. The LED light reflected by the reflective surface is reflected again by the substrate 30 or the solid-state light emitting element 31 toward the opening 42 or the cover member 40. In this way, by being repeatedly reflected by the cover member 40, the substrate 30, and the solid-state light emitting element 31, the LED light eventually passes through the opening 42, and is emitted from the first light source unit 111.

Typically, in order to efficiently capture LED light emitted over a wide angle range with a collimator lens, the diameter of the collimator lens needs to be large. In contrast, according to the present embodiment, even if the LED light has a large emission angle, it has been converted to LED light with a small emission angle when it is emitted from the opening 42, as a result of being reflected multiple times within the first light source unit 111. For that reason, the present embodiment can improve light use efficiency regarding LED light without increasing the diameter of the collimator lens 121.

In addition, in the first light source unit 111 according to the present embodiment, the area of the opening 42 of the cover member 40 is set to be smaller than the area of the light emitting surface 31A. The smaller the light emitting surface is, the smaller the etendue (the product of the area of the light emitting surface and the emission solid angle) of the LED light is. Furthermore, the smaller the etendue of the LED light is, the higher the light use efficiency of the optical system including the collimator lens 121 is. Therefore, the light use efficiency regarding LED light can be further improved by using the cover member 40, which has the opening 42 with a smaller area than that of the light emitting surface 31A.

As shown in FIG. 6, the third light source unit 113 includes a solid-state light emitting element 31 that emits blue LED light and a phosphor 32. The phosphor 32 is disposed on the light emitting surface 31A of the solid-state light emitting element 31 so as to cover the entire light emitting surface 31A. The cover member 40 of the third light source unit 113 is used to improve the light use efficiency of the optical system regarding LED light and fluorescent light, and the luminous efficiency of the phosphor 32. Some of the LED light emitted from the solid-state light emitting element 31 is used to excite the phosphor 32 while the other passes through the phosphor 32. This causes both LED light and fluorescent light to be emitted from the solid-state light emitting element 31 provided with the phosphor 32. As with LED light in the first light source unit 111, the LED light and the fluorescent light pass through the opening 42 as a result of being reflected multiple times within the third light source unit 113. In this way, light having a large emission angle is converted to light having a small emission angle while the etendues of LED light and fluorescent light is reduced. Thus it is possible to improve light use efficiency regarding LED light and fluorescent light.

Also, in the third light source unit 113, LED light that passes through the phosphor 32 and is reflected within the third light source unit 113 multiple times, is partially incident on the phosphor 32 again. Then, some of the LED light that has been incident again on the phosphor 32 is used to excite the phosphor 32. Thus, it is possible to improve emission efficiency of the phosphor 32.

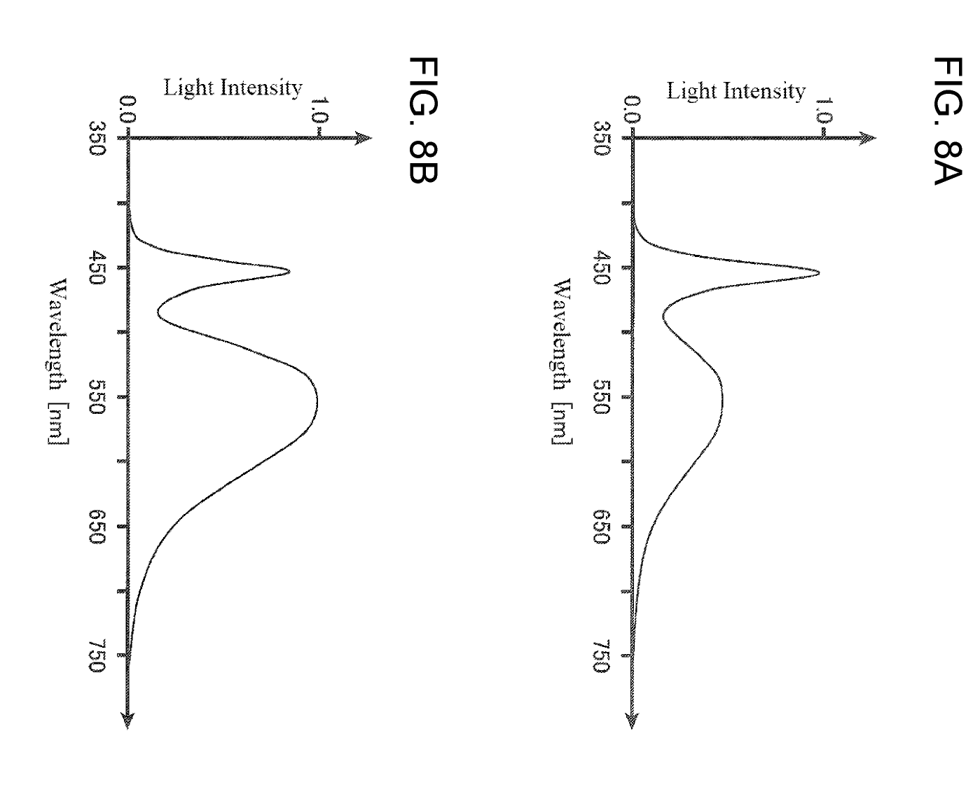

FIG. 8 shows the spectral intensity distribution of light emitted from the third light source unit 113. FIG. 8(a) shows the spectral intensity distribution in the case where the third light source unit 113 does not include a cover member 40, and FIG. 8(b) shows the spectral intensity distribution in the case where the third light source unit 113 includes a cover member 40. In FIG. 8, the horizontal axis of the spectral intensity distribution indicates the wavelength (nm), and the vertical axis indicates the intensity of the light L. Note that the vertical axis is standardized such that the maximum intensity value is 1.

Some of the blue LED light emitted from the solid-state light emitting element 31 is used to excite the phosphor 32 while the other passes through the phosphor 32. For that reason, as shown in FIG. 8(a), the light emitted from the third light source unit 113 has a spectral intensity distribution with two peaks, a peak wavelength of blue LED light and a peak wavelength of fluorescent light. In addition, if the cover member 40 is used, some of the blue LED light that passes through the phosphor 32 is used to excite the phosphor 32. For that reason, as shown in FIG. 8(b), the proportion of fluorescent light in the spectral intensity distribution of the light emitted from the third light source unit 113 is larger than that in the case without a cover member 40. Also, while the fluorescent light of the light emitted from the third light source unit 113 is used to illuminate the subject, the blue LED light is not used to illuminate the subject. Accordingly, the amount of the fluorescent light can be increased by using the cover member 40, and accordingly the amount of light of the illumination light L can be increased.

Illustrative embodiments of the present invention have been described above. The embodiments of the present invention are not limited to those described above, and various changes can be made without departing from the scope of the technical idea of the present invention. For example, appropriate combinations of embodiments and the like explicitly given as examples in this specification and obvious embodiments and the like are also encompassed in embodiments of the present invention.

In the electronic endoscope system 1 shown in FIG. 1, although the light source device 201 is provided in the processor 200, it may be configured as a separate device from the processor 200 and the electronic endoscope 100. Also, according to one embodiment, the light source units 111 to 114 may be incorporated into the electronic endoscope 100 as light source devices. In this case, according to one embodiment, it is preferable that the light source units 111 to 114 are incorporated into the connection portion 104 connected to the processor 200. Such incorporation into the connection portion 104 can prevent poor connection caused during connecting work.

Also, according to one embodiment, the light source units 111 to 114 may be incorporated into the distal end portion 106, in which orientation light distribution lens 12 of the electronic endoscope 100 is provided. If the light source units 111 to 114 are incorporated into the distal end portion 106, the LCB 11 is no longer necessary. Therefore, the illumination light is not affected by the transmission characteristics of the LCB 11 and the diameter of the portion inserted into the body cavity is narrowed. Thus, it is possible to reduce stress on the subject.

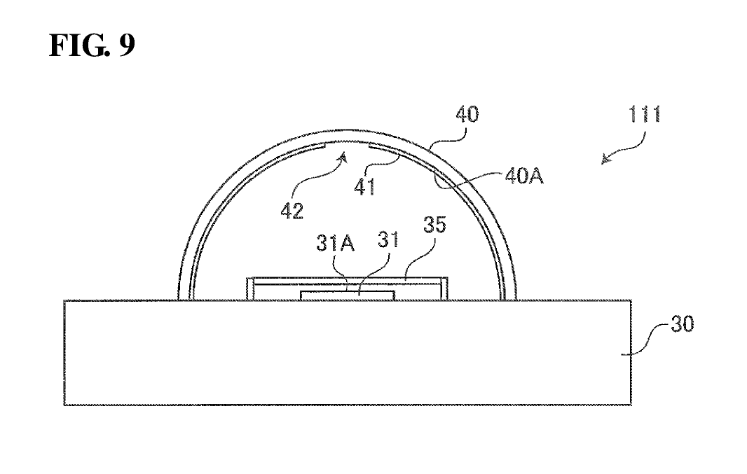

According to one embodiment, the opening 42 of the cover member 40 does not have to be a through hole provided in the cover member 40. According to one embodiment, the opening 42 preferably has a characteristic of allowing LED light and fluorescent light to pass through it. FIG. 9 is a cross sectional view of a first light source unit 111 according to one embodiment. In this variation, the cover member 40 is formed by molding a light-transmitting base plate made of material that allows light to pass therethrough (for example, glass or light-transmitting resin material), so as to have a hemispherical shell shape. A reflective film 41 is formed on the inner wall surface 40A of the cover member 40 except for a part of its region that is opposite the light emitting surface 31A. The region of the inner wall surface 40A where the reflective film 41 is not formed is an opening 42, which allows LED light to pass therethrough.

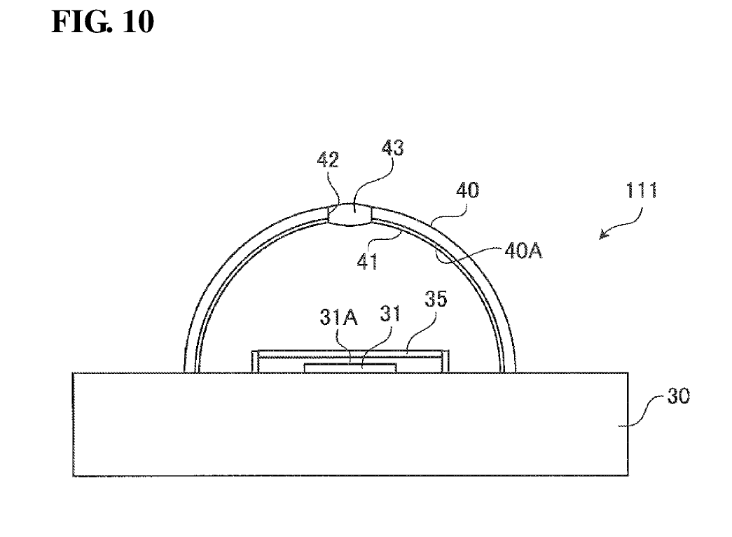

According to one embodiment, a lens is preferably disposed in the opening 42 of the cover member 40. FIG. 10 shows a cross sectional view of a first light source unit 111 that includes a convex lens 43. In the configuration shown in FIG. 10, the convex lens 43 is disposed in the opening 42. The emission angle of light emitted through the opening 42 is narrowed by the convex lens 43. As a result, the collimator lens 121 can easily capture the light emitted from the first light source unit 111, and thus it is possible to improve light use efficiency regarding the illumination light L. Note that the convex lens 43 need not be disposed in the opening 42; it may be disposed outside the opening 42 so as to cover the opening 42.

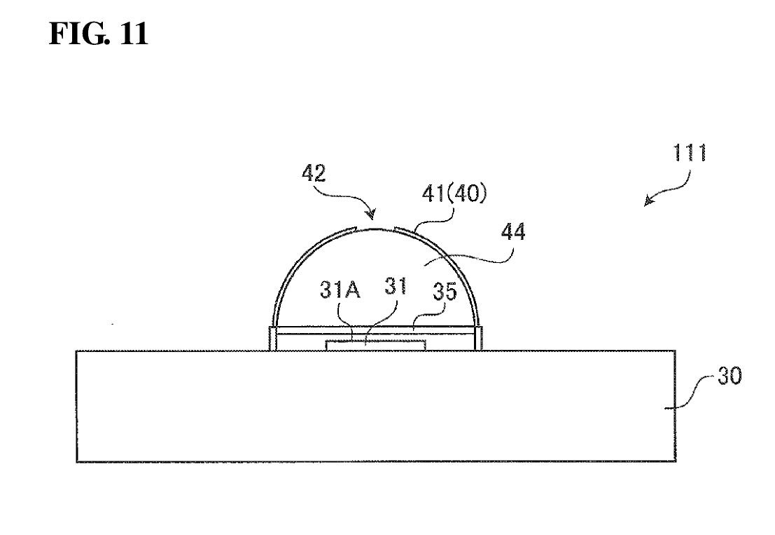

In addition, according to one embodiment, the cover member 40 need not be hollow. FIG. 11 shows a cross sectional view of a first light source unit 111 according to one embodiment. According to this embodiment, preferably, a lens 44 is disposed on the cover glass 35. The lens 44 is a plano-convex lens disposed such that the flat surface of the lens 44 is opposite the cover glass 35. For example, a hemispherical lens is used as the plano-convex lens. The cover member 40 is a reflective film 41 formed on the outer convex surface of the lens 44 except for a part of the region that is opposite the light emitting surface 31A. The region of the convex surface of the lens 44 where the reflective film 41 is formed is the reflective surface that reflects LED light. The region of the spherical surface of the lens 44 where the reflective film 41 is not formed is an opening 42, which allows LED light to pass therethrough. According to one embodiment, while the cover member 40 is not hollow, the cover member 40 (the reflective film 41) is disposed to be spaced apart from the light emitting surface 31A across the light-transmitting lens 44. For that reason, the LED light emitted from the light emitting surface 31A can be reflected multiple times within the first light source unit 111 and converted to LED light having a small emission angle.

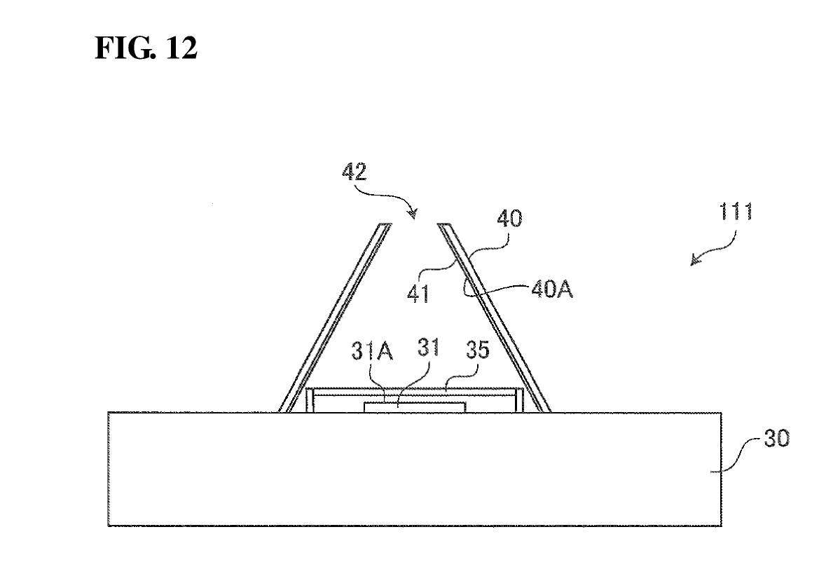

According to one embodiment, it is also preferable for the cover member 40 to have a shape other than a dome shape. FIG. 12 shows a cross sectional view of a first light source unit 111 according to one embodiment. In this embodiment, it is preferable for the cover member 40 to have a prismatic shape. According to one embodiment, the cover member 40 preferably has a truncated pyramid shape that has a cross-sectional area that gradually decreases in a direction away from the solid-state light emitting element 31. Furthermore, as described above, the area of an end of the opening 42 of the cover member 40, which is opposite to its end at which the solid-state light emitting element 31 is disposed, is set to be smaller than the area of the light emitting surface 31A. Furthermore, a reflective film 41 is formed on the inner wall surface 40A of the cover member 40. In this way, even if the cover member 40 is formed in a shape other than a dome shape, light having a large emission angle emitted from the solid-state light emitting element 31 is converted to light having a small emission angle while the etendue of the LED light is reduced. Also, according to one embodiment, the cover member 40 preferably has the shape of a truncated cone, not a truncated pyramid.

In the above-described embodiments, while a metallic multilayer film or a dielectric multilayer film is used as the reflective film 41 formed on the cover member 40, the reflective film is not limited to this configuration, according to one embodiment. According to one embodiment, the reflective film 41 preferably has a characteristic of diffusely reflecting light incident thereon. If the reflective film 41 is a metallic multilayer film or a dielectric multilayer film, light incident on the reflective film 41 is regularly reflected (specularly reflected). Therefore, to convert light having a large emission angle to light having a large emission angle, the light needs to be reflected multiple times by the reflective film 41, the substrate 30, and the solid-state light emitting element 31, etc. However, if the reflective film 41 has a characteristic of diffusely reflecting light, at least some of the light incident on the reflective film 41 is converted to light having a small emission angle at a single reflection. Therefore, compared with the case that involves multiple reflections, it is possible to reduce the proportion of light absorbed by the reflective film 41 and the substrate 30. According to one embodiment, the reflective film 41 preferably has a roughened surface to have a characteristic that realizes diffuse reflection.

LEDs are envisioned as the solid-state light emitting elements 31 in the above embodiments. The present invention is not limited to this, and it is also preferable that LDs (Laser Diodes) are employed as the solid-state light emitting elements 31.

### EXPLANATION OF REFERENCE NUMERALS

1 electronic endoscope system
11 LCB
12 light distribution lens
13 objective lens
14 solid-state image sensor
15 driver signal processing circuit
16 memory
21 system controller
22 timing controller
23 memory
24 operation panel
25 condensing lens
26 pre-stage signal processing circuit
27 image memory
28 post-stage signal processing circuit
30 substrate
31 solid-state light emitting element (LED)
31A light emitting surface
32 phosphor
35 cover glass
40 cover member
40A inner wall surface
41 reflective film
42 opening
43 convex lens
44 lens
100 electronic endoscope
104 connection portion
105 illumination light emission opening
106 distal end portion
111-114 lighting source unit
121-124 collimator lens
141-144 light source drive circuit
131-133 dichroic mirror
200 processor
201 light source device

1. An endoscope light source device comprising:
a solid-state light emitting element configured to emit light from a light emitting surface thereof; and
a cover member that covers the solid-state light emitting element such that the cover member is spaced apart from the light emitting surface,
wherein the cover member includes
a reflective surface configured to reflect the light emitted from the light emitting surface, and
an opening configured to emit some of the light emitted from the light emitting surface and some of the reflected light reflected by the reflective surface.

2. The endoscope light source device according to claim 1, wherein the area of the opening is smaller than the area of the light emitting surface.

3. The endoscope light source device according to claim 1, wherein the cover member has a hollow dome shape.

4. The endoscope light source device according to claim 1, wherein the cover member includes a light-transmitting substrate configured to allow the light emitted from the light emitting surface to pass therethrough,
wherein the reflective surface is a region of a surface of the light-transmitting substrate where a reflective film configured to reflect the light emitted from the light emitting surface is formed, and
wherein the opening is a region of the surface of the light-transmitting substrate where the reflective film is not formed.

5. The endoscope light source device according to claim 1, comprising a convex lens disposed in the opening.

6. The endoscope light source device according to claim 1, wherein the cover member includes a plano-convex lens disposed such that a flat surface thereof is opposite the light emitting surface,
wherein the reflective surface is a region of a convex surface of the plano-convex lens where a reflective film is formed, and
wherein the opening is a region of the convex surface where the reflective film is not formed.

7. The endoscope light source device according to claim 1, comprising a protective member that protects the light emitting surface,
wherein the protective member is
disposed to cover the light emitting surface, and
configured to allow the light emitted from the light emitting surface to pass therethrough, and
wherein the cover member is disposed to cover the solid-state light emitting element and the protective member.

8. The endoscope light source device according to claim 1, comprising a phosphor disposed between the light emitting surface and the reflective surface and configured to absorb some of the light emitted from the light emitting surface and emit fluorescent light.

9. The endoscope light source device according to claim 8, wherein the phosphor is disposed on the light emitting surface.

10. An endoscope system comprising:
an endoscope light source device that includes a first light source unit including a first solid-state light emitting element configured to emit first light from a first light emitting surface, and a first cover member that covers the first solid-state light emitting element such that the first cover member is spaced apart from the first light emitting surface, the first cover member including a first reflective surface configured to reflect the first light emitted from the first light emitting surface and a first opening configured to emit some of the first light and some of the reflected light reflected by the first reflective surface; and
an endoscope that includes a connection portion connected to the endoscope light source device and a distal end portion having an illumination light emission opening configured to emit illumination light transmitted through an optical cable from the connection portion to illuminate a subject.

11. An endoscope system according to claim 10, wherein the endoscope light source device includes, in addition to the first light source unit,
a second light source unit including a second solid-state light emitting element configured to emit second light from a second light emitting surface thereof, and a second cover member that covers the second solid-state light emitting element such that the second cover member is spaced apart from the second light emitting surface, the second cover member including a second reflective surface configured to reflect the second light and an opening configured to emit some of the second light and some of the reflected light reflected by the second reflective surface, and the second light source unit including a phosphor disposed between the second light emitting surface and the second reflective surface and configured to absorb some of the second light and emit fluorescent light; and
an optical element provided on a light path of the first light and a light path of the second light and the fluorescent light and configured to extract the fluorescent light from the second light and the fluorescent light and emit combined light on a light path that combines the light path of the first light and the light path of the fluorescent light.

12. An endoscope system according to claim 11, wherein the phosphor includes material configured to emit a light component having a wavelength band of 460 to 600 nm.

13. An endoscope comprising:
a light source unit including a solid-state light emitting element configured to emit light from a light emitting surface thereof, and a cover member that covers the solid-state light emitting element such that the cover member is spaced apart from the light emitting surface, the cover member including a reflective surface configured to reflect the light emitted from the light emitting surface, and an opening configured to emit some of the light and some of the reflected light reflected by the reflective surface; and
a distal end portion having an illumination light emission opening configured to emit the light emitted by the light source unit as illumination light to illuminate a subject.
